(19) United States
(12) Patent Application Publication
(10) Pub. No.: US 2005/0131224 A1
(43) Pub. Date: Jun. 16, 2005

(54) Method for preparing radiolabeled thymidine

(75) Inventors: Joseph C. Walsh, Pacific Palisades, CA (US); Henry C. Padgett, Hermosa Beach, CA (US)

Correspondence Address: PITTS AND BRITTIAN P C, P O BOX 51295, KNOXVILLE, TN, 37950-1295, US

(73) Assignee: CTI PET Systems, Inc.

(21) Appl. No.: 10/736,084

(22) Filed: Dec. 15, 2003

(51) Int. Cl. C07H 19/06

(52) U.S. Cl. 536/28.4

(57) ABSTRACT

The invention is a novel method and precursor for preparing radiolabeled nucleosides. In particular, the invention is useful for preparing 3′-[¹⁸F]fluorothymidine. The method uses an enol group that is attached to the 2-position on the pyrimidine ring. The enol group attenuates thymidines activity so that the thymidine is easily radiolabeled in short number of steps with good yield.

### BACKGROUND OF THE INVENTION

Positron emission tomography (PET) is a diagnostic imaging technique for measuring the metabolic activity of cells in the human body. PET can show images of blood flow, glucose metabolism in the brain, or rapid changes in activity in various areas of the body. It can be used to show changes in physiology before any change in gross anatomy has occurred. PET has been used in diagnosing diseases such as cancer, heart disease, Alzheimer's disease, Parkinson's disease, and schizophrenia.

PET uses chemical compounds that are labeled with radioactive atoms that decay by emitting positrons. The most commonly used PET radioisotopes are ¹¹C, ¹³N, ¹⁵O, and ¹⁸F. Typically, the labeled compound is a natural substrate, substrate analog, or drug that is labeled with a radioisotope without altering the compound's chemical or biological properties. After injection into the tissue, the radiolabeled compound should follow the normal metabolic pathway of its unlabeled counterpart. The labeled compound emits positrons as it moves through the tissue. Collisions between the positrons and electrons that are present in the tissue emit gamma rays that are detectable by a PET scanner.

Radiolabeled thymidine is a PET tracer that is useful for imaging tumors. In particular, 3′-Deoxy-3′-[¹⁸F]-fluoro-thymidine (¹⁸F-FLT) has been used for visualizing DNA replication in humans and animals. ¹⁸F-FLT is incorporated into DNA during the synthesis phase of the cell cycle and therefore is a useful indicator of cellular proliferation.

After injection into a patient, ¹⁸F-FLT is taken up by cells and undergoes phosphorylation by thymidine kinase-1 (TK), an enzyme that is expressed during cellular DNA synthesis. The phosphorylated FLT molecule is retained within the cell, which results in its accumulation. As a result, ¹⁸F-FLT provides insight into cellular activity and is an excellent proliferation marker for PET tumor studies.

The usefulness of ¹⁸F-FLT as a tumor imaging agent has resulted in a need to develop methods for its quick and efficient synthesis. Typical methods for preparing ¹⁸F-FLT have low reaction yields. A common problem associated with preparing ¹⁸F-FLT has been the incorporation of ¹⁸F into the thymidine. Recent articles have reported that in order to get acceptable yields of ¹⁸F-FLT it is necessary to mask the reactivity of the NH moiety on the pyrimidine ring (Grierson, J. R., Shields, A. F., Nuclear Medicine and Biology, 2000, 27 143-156; Eisenhut, et al, Nuclear Medicine and Biology, 2002, 29, 263-273).

The imide functionality on the pyrimidine ring in thymidine can adopt a tautomeric form in solution. When in this tautomeric state, the oxygen becomes increasingly nucleophilic. As a result, the 2-O group can displace leaving groups anti to the 2-O group to produce 2,3′-anhydrothymdine. To prevent the formation of this tautomer, and thus prevent the unwanted formation of 2,3′-anhydrothymidine, recent methods have attached protecting groups, such as alkyl and acetyl, to the 3-N group on the pyrimidine ring. When alkylated, the nitrogen is unable to participate in tautomer formation. As a result, these methods have produced ¹⁸F-FLT in higher yields than methods using unalkylated 3-N precursors.

Although recent methods have increased yield by alkylating 3-N group on the pyrimidine ring, the final product yield is often reduced during the deprotection step. This method of preparing ¹⁸F-FLT typically takes 7 steps and results in yields that are about 20% after deprotection. A more recent method discloses shielding the 3-N amine with t-butyloxycarbonyl. This method results in a process that takes 6 steps and has a yield that is about 31% after deprotection.

### BRIEF SUMMARY OF THE INVENTION

The invention is a novel method for preparing radiolabeled nucleosides. More specifically, the invention is particularly useful for preparing ¹⁸F-FLT and a related precursor. The method allows the synthesis of ¹⁸F-FLT in a short number of steps with good yield.

The method uses a novel protecting group on the pyrimidine base to attenuate the pyrimidine's reactivity. The ¹⁸F-FLT precursor is prepared using a synthetic route that is short and simple. The novel precursor is prepared by replacing a carbonyl group on the pyrimidine base with a 2-O-alkyl moiety to produce a thymidine intermediate having an enol ether moiety.

The method begins with 2,3′-anhydrothymidine or thymidine that has been converted into 2,3′-anhydrothymidine. In the next step, the 5′-hydroxy group is protected. After the 5′-hydroxy group is protected, the carbonyl group located at the 2-position on the pyrimidine base is enolated. Typically, the enolating step comprises reacting 2,3′-anhydrothymidine with a reagent that opens the 2,3′-anhydro-ring and attaches to the carbon at the 2-position. In the final step, converting the 3′-hydroxy group into a leaving group activates the thymidine compound. The ¹⁸F-FLT precursor has the following formula: 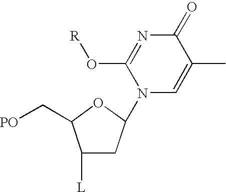
wherein R is an alkoxy blocking group, P is a hydroxyl protecting group, and L is a leaving group.

The precursor is immediately ready for radiolabeling or alternatively can be stored for future use. After radiolabeling, the protecting group and alkyl group are removed. Typically, the removal is carried out with acid hydrolysis.

### BRIEF DESCRIPTION OF THE SEVERAL VIEWS OF THE DRAWING(S)

Having thus described the invention in general terms, reference will now be made to the accompanying drawings and wherein:

FIG. 1 illustrates a reaction scheme for preparing ¹⁸F-FLT;

FIG. 2 is structural drawing of thymidine showing the numbering of carbon atoms in the compound; and

FIG. 3 illustrates two alternative reaction schemes for preparing an ¹⁸F-FLT precursor.

The invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

### DETAILED DESCRIPTION OF THE INVENTION

The synthesis of radiolabeled nucleosides can begin with a nucleoside having a pyrimidine base, such as thymidine, uridine, or cytidine. In the invention, the nucleoside with a pyrimidine base is converted into an anhydronucleoside.

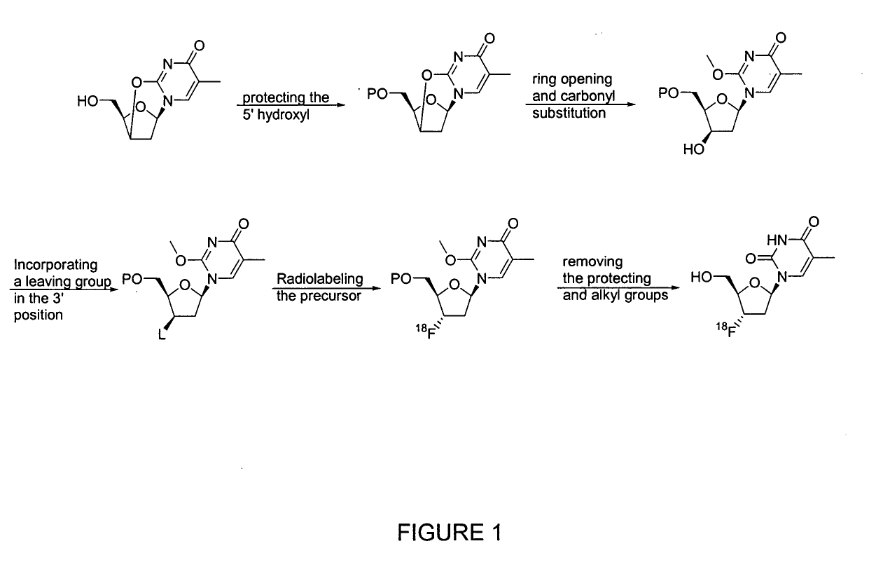

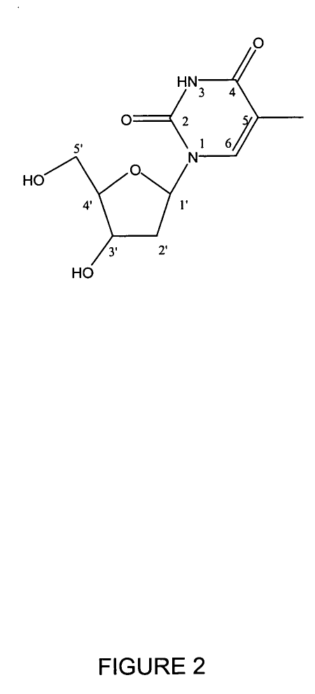

FIG. 1 illustrates an exemplary reaction scheme for a method of preparing ¹⁸F-FLT in accordance with the invention. For ease of discussion, FIG. 2, illustrates a thymidine molecule in which the carbon atoms have been numbered. The numbering convention shown in FIG. 2 is used throughout the disclosure. It should be recognized that the carbon atoms could be numbered differently and that the invention is not limited by any particular numbering format.

The synthesis of ¹⁸F-FLT and its related precursor can begin with thymidine. In the first step, thymidine is converted into 2,3′-anhydrothymidine to produce a compound having the following formula: 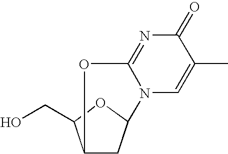
2,3′-anhydrothymidine can be a useful starting compound because the 3′-hydroxyl group is in the beta position so that a leaving group can also be positioned in the beta position in a subsequent reaction. As a result, when fluorinated, ¹⁸F can attack the 3′-carbon from the anti direction.

There are a variety of known techniques for converting a nucleoside into an anhydronucleoside. For example, the anhydronucleoside can be prepared by mixing thymidine with triphenylphosphine and azeotropically drying with portions of acetonitrile (MeCN). The resulting mass is suspended in MeCN and then cooled. The mixture is rapidly stirred and diisopropylazodicarcobxylate in MeCN is added dropwise to the mixture. The resulting mixture is treated with water to form a suspension that is filtered to afford anhydrothymidine. (Grierson, J. R., Shields, A. F., Nuclear Medicine and Biology, 2000, 27 143-156; Balagopala, M. I., Ollapally, A. P., and Lee, H. J., Nucleosides-Nucleotides, 1996, 15(4) 899-906).

U.S. Pat. No. 5,717,086 discloses a method of converting a nucleoside into a 2,3′-anhydronucleoside by reacting it with a dehydrating agent in the presence of an acid. Specifically, it discloses that 2′-deoxyuridine may be reacted with a combination of diisopropylazodicarboxylate (DIAD) or diethylazodicarboxylate (DEAD) and a triaryl- or trialkyl-phosphine or -phosphite, e.g. triphenylphosphine, preferably in the presence of an acid, in an inert polar solvent. It should be recognized that there are many different methods that can be used to convert a nucleoside into its anhydronucleoside derivative, although not necessarily with equivalent results.

Alternatively, the synthesis may begin with a commercially available 2,3′-anhydro nucleoside, such as 2,3′-anhydrothymidine or one of its derivatives. It should be recognized that the invention can also include nucleosides derivatives that contain additional substituents provided that the substituents are non-interfering and do not prevent, block, or negatively impact the reactivity or functionality of the precursor, any reaction steps, or the final product. Such derivatives are known in the art and include, without limitation, deuterated derivatives, such as 2′-deuterated nucleosides, or derivatives having different substituents at the 5-position, such as hydro, bromomethyl, benzyl, or the like.

After converting thymidine into 2,3′-anhydrothymidine, the 5′-position is protected with a hydroxyl protecting group to produce a compound having the following formula: 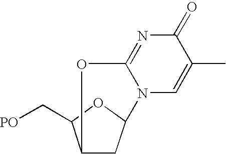
wherein P is a hydroxyl protecting group.

Hydroxyl protecting groups that are useful in the invention must fulfill a number of requirements. The protecting groups should react selectively in good yield to give a protected substrate that is stable for future reactions. The protecting groups should be able to be selectively removed in good yield at the end of the reaction scheme or at any other time that is appropriate. Suitable protecting groups should not be affected by reaction conditions and should not interfere with reactions on other portions of the molecule. It is also desirable that the protecting groups will help enhance reactions by increasing yield or selectivity.

Hydroxyl protecting groups that are useful in the invention include, without limitation, ethers including, without limitation, methoxymethyl ether, methylthiomethyl ether, 2-methoxyethoxymethyl ether, 1-ethoxyethyl ether, 1-methyl-1-methoxyethyl ether, t-butyl ether, allyl ether, benzyl ether, 4-nitrobenzyl ether, o-nitrobenzyl ether, trityl ether, monomethoxytrityl ether, dimethoxytrityl ether, and tritylone ether; cyclic ethers including, without limitation, tetrahydropyran ether, tetrahydrothiopyranyl ether, 4-methoxy tetrahydropyran ether, 4-methoxytetrahydrothiopyranyl ether, tetrahydrofuran ether, and tetrahydrotriofuranyl ether; esters including, without limitation, isobutyrate ester, pivaloate ester, adamantoate ester, benzoate ester, and 2,4,6,-trimethylbenzoate ester; carbonates including, without limitation, methyl carbonate, allyl carbonate, benzyl carbonate, p-nitrobenzyl carbonate, t-Bu carbonate, and S-benzylthio carbonate; N-phenyl carbamate; and nitrate ester.

Hydroxyl protecting groups that are particularly useful include, without limitation, dimethoxytrityl (DMTr), monomethoxytrityl (MMTr), trityl (Tr), t-butyloxycarbonyl (“boc”), t-butyldimethylsilyl (TBDMS), t-butyldiphenylsilyl (TBDPS), tetrahydropyranyl ether, tetrahydrofuranyl ether, ethoxyethyl ether, and 1-methyl-1-methoxyethyl ether. It should be recognized that a variety of different protecting groups can be used in the practice of the invention, although not necessarily with equivalent results.

In the next step, the 2,3′-anhydro ring is opened and the carbonyl located at the 2-position on the pyrimidine ring is substituted with an enol group. This can be accomplished by reacting the protected 2,3′-anhydrothymidine derivative with a reagent, such as an alkoxide, that opens the 2,3′-anhydro-ring and attaches to the 2-position to form the following compound: 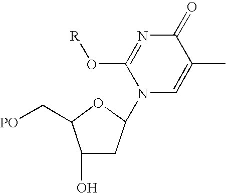
wherein P is the same as defined above and R is an alkoxy blocking group.

Typically, R is alkyl C₁-C₄, i-propyl, benzyl, cycloalkane C₃-C₆, phenyl, tosyl, acetate, or benzoate. Reagents that are useful for this step will typically be able to both open the ring and enolate the pyrimidine base. The ring-opening/enolating reagent should not react with other sites or moieties on the thymidine, should maintain stability throughout the fluorination process, and should be easily removed in an acidic environment. Suitable reagents for opening the 2,3′-anhydro ring and enolating the pyrimidine base include, without limitation, alkoxides with alkyl C₁-C₄, such as sodium methoxide or sodium ethoxide, or the like. Typically, the alkoxide will open the ring and attach to the 2-position to form the enol moiety. Other useful reagents include alkoxides that have alkyl groups such as i-propyl, benzyl, cycloalkoxides C₃-C₆, phenoxide, tosyate, acetate, and benzoate.

Typically the enol group is a vinylic heteroatom moiety that can generate a carbonyl upon hydrolysis. The 2-enol ether group does not have to contain oxygen and can contain other heteroatom moieties, such as 2-thio or a 2-amino derivative. An alternative embodiment of the enolated intermediate has the following formula: 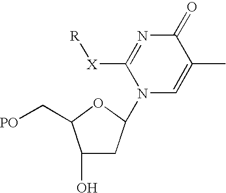
wherein P and R are the same as defined above and X is oxygen, nitrogen, or sulfur.

Opening the ring and enolating the 2-carbon in a single step provides a reaction mechanism that is completed in fewer steps with less time and expense. Enolating the 2-thymidine intermediate also masks the reactivity of the 3-N amine and prevents the formation of the nucleophilic tautomer. As a result, 2,3′-anhydrothymidine is not produced as a byproduct and ¹⁸F-FLT yield is increased. In addition, small alkyl groups, such as methyl and ethyl, can be used to mask the amine group so that it is not necessary to use larger protecting groups. As a result, precursors in accordance with the invention have a slightly lower molecular weight, which means that less material is needed to perform the fluorination step.

Alternatively, the ring can be opened in one step using a base, such as sodium hydroxide or tetrabutylammonium hydroxide to produce beta-thymidine followed by a second step that alkylates the 4-carbonyl.

In the next step of preparing the ¹⁸F-FLT precursor, a leaving group is incorporated at the 3′-position to produce the precursor having the following formula: 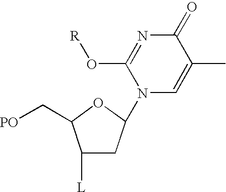
wherein P and R are the same as defined above and L is a leaving group.

The leaving group activates the thymidine derivative and is replaced during the [¹⁸F] fluorination step. During the radiolabeling step, [¹⁸F] fluoride attacks the 3′-carbon atom anti to the 3′-leaving group resulting in substitution of the leaving group by a bimolecular nucleophile substitution mechanism (S_{N}2).

Leaving groups that are useful in the invention are moieties that can be displaced from the 3′-carbon atom by nucleophilic substitution. The leaving group should attach to the 3′-hydroxyl or replace it to form a leaving group at the 3′-postion. The leaving group moiety should not react with other sites or functional groups that may be present on the thymidine derivative. The leaving group should also be able to be quickly replaced by the radioisotope during the radiolabeling step. Typically, the leaving group should be replaced by ¹⁸F in polar aprotic solvent.

The term leaving group (“L”) refers to moieties that should be susceptible to displacement by a nucleophile, wherein the 3′-hydroxy can attach to another substituent directly to form a leaving group or the 3′-hydroxy may be removed in order to incorporate the leaving group. Sulfonate ester is an exemplary leaving group that is formed from a sulfonyl moiety attaching directly to the 3′-hydroxy.

Useful leaving groups that combine with the 3′-hydroxy include, without limitation, sulfonyl moieties, such as alkylsulfonyl, substituted alkylsulfonyl, arylsulfonyl, substituted arylsulfonyl, hetercyclcosulfonyl or trichlorcacetimidate groups. Particularly useful groups include, without limitation, benzenesulfonyl, methylsulfonyl (mesylate), p-methylbenzenesulfonyl (tosylate), 4-nitrobenzene sulfonyl (nosylate), p-bromobenzenesulfonyl, trifluoromethylsulfonyl (triflate), trichloroacetimidate, 2,2,2-trifluoroethanesulfonyl, and imidazolesulfonyl. It should be recognized that other moieties can be used to form —O-L′ leaving groups, although not necessarily with equivalent results.

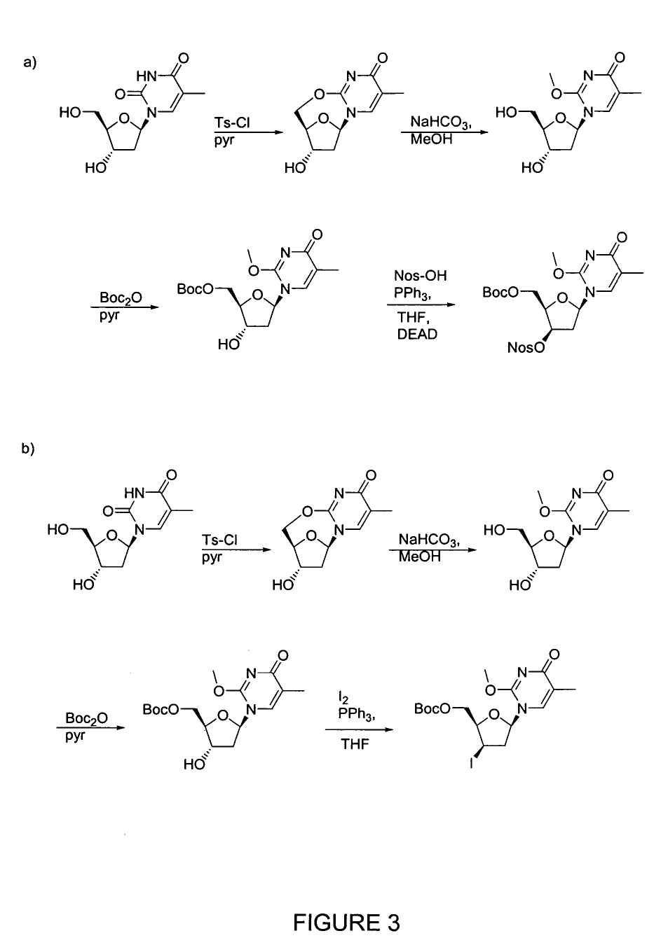

Other useful ¹⁸F-FLT precursors include thymidine derivatives wherein the 3′hydroxy has been completely replaced with an alternative leaving group, such as a halogen. In this manner, a reaction scheme is illustrated in FIG. 3 in which the 3′hydroxy group has been substituted with halogen, such as iodine.

The precursor is now ready for immediate [¹⁸F]fluorination or can be stored and transported for future use. The precursor is shelf stable and is highly reactive with ¹⁸F in polar aprotic solvents.

Radiolabeling can be carried out using a variety of methods. In the method for preparing ¹⁸F-FLT, the precursor is treated with Kryptofix 222′ and potassium carbonate in the presence of a polar aprotic solvent (Wodarski, C., et al., J. Labelled Cpd. Radiopharm., 2000, 43 1211-1218; Blocher A., et al., J. of Radioanalytical and Nuc. Chemistry, 2002, 251(1), 55-58; Martin, S. J. et al., Nuclear Medicine and Biology, 2002, 29 263-273). Useful solvents include, without limitation, acetonitrile, pyridine, N,N-dimethylformamide (DMF), dimethyl sulfoxide (DMSO), and blends thereof.

In the final step of preparing ¹⁸F-FLT, the 2-0 alkyl group and 5′-hydroxy protecting groups are removed. Typically, the alkyl and protecting group are removed by hydrolyzing the radiolabeled nucleoside. Useful hydrolyzing reagents include, without limitation, acids such as HCl, HBr, HOAc, H_{2 SO}₄, HI, trimethylsilyliodide (TMSI), and H₃PO₄.

As should be evident from the above disclosure, the method would also be useful for preparing other radiolabeled nucleoside compounds that have a pyrimidine base. For example, the method could be used to prepare radiolabeled derivatives of uridine and cytidine. In preparing radiolabeled cytidine and uridine derivatives, the synthesis should begin with their 2′-deoxy derivatives.

### EXAMPLE

### Synthesis of 5′-O-DMT-3′-methane sulfonyl-3-O-Me-thymidine

Step a: Synthesis of Anhydrothymidine.

To a dried round bottom flask containing thymidine (10.0 g, 41.6 mmol) was added triphenylphosphine (21.8 g, 82.2 mmol) and acetonitrile (CH₃CN) (160 mL). The suspension was cooled to −20° C. (40:60 iPrOH:H₂O) and dry ice). To the reaction mixture was added diisopropylazodicarboxylate (16.8 g, 82.2 mmol) as CH₃CN solution (60 mL) dropwise via an addition funnel, over a period of 1 hour. After the addition, the mixture was stirred for an additional 90 minutes at 20° C. The mixture was allowed to warm to 10° C. over a period of 5 hours. The reaction was then quenched with H₂O (6 mL) forming a white suspension. The reaction was allowed to stand for 30 minutes and then was filtered. The collected solid was washed with cold CH₃CN and dried under vacuum to afford 7.7 g (83% yield) of a white solid. If desired, the solid may be recrystallized in ethanol.

Step b: Synthesis of 5′-O-DMT-3′-anhydrothymidine.

To a round bottom flask containing 2,3′-anhydrothymidine (600 mg, 2.7 mmol) and pyridine (10 mL) was added (dimethoxytrityl chloride (DMT-Cl) (1.8 g, 5.3 mmol). The reaction was stirred at room temperature for 3 hours. The reaction was then poured onto H₂O and extracted into ethyl acetate (EtOAc) (3x's). The combined organics were washed with brine (5x's), dried (MgSO₄), filtered and concentrated to dryness. The residue was purified by column chromatography on silica gel using EtOAc to remove byproducts followed by 5% methanol:methylene chloride (MeOH:CH₂Cl₂) to afford 1.17 g (83%) of a white solid whose spectral data matches reported values.

Step c: Synthesis of 5′-O-DMT-3′-hydroxy-2-O-Me-thymidine.

To a round bottom flask containing 5′-O-DMT-3′-anhydrothymidine (1.17 g, 2.2 mmol) was added MeOH (20 mL). Sodium methoxide (NaOMe) (1.25 g, 22.3 mmol) was added and the reaction stirred at room temperature for 48 hours. The reaction was then poured onto saturated ammonium chloride (NH₄Cl) and was extracted into CH₂Cl₂ (3x's). The combined organics were washed with brine (3x's), dried (magnesium sulfate (MgSO₄)), filtered and concentrated to dryness. The residue was purified using column chromatography on silica gel using EtOAc to remove byproducts followed by 5% MeOH:CH₂Cl₂ to afford 450 mg (36%) of a white solid. Alternatively, this compound may be recrystalized from CH₃CN. MS: Calc'd for C₃₂H₃₄N₂O₇: 558.24; found: 559 (M+H)

¹H NMR (300 MHz, CD₃CN) δ: 1.72 (3H, s), 2.54-2.62 (1H, m), 3.32-3.52 (3H, m), 3.78 (6H, s), 3.93 (3H, s), 4.14-4.19 (1H, m), 4.31-4.33 (1H, m), 6.09 (1H, dd, J=7.9, 1.8 Hz), 6.87-6.90 (4H, m), 7.25-7.51 (9H, m), 7.62 (1H, s).

¹³C NMR (75 MHz, CD₃CN) δ: 13.99, 42.26, 55.81, 55.90, 63.68, 70.75, 85.21, 87.20, 87.38, 114.08, 116.49, 127.84, 128.85, 129.03, 131.01, 135.31, 136.94, 137.00, 146.11, 156.43, 159.71.

Step d: Synthesis of 5′-O-DMT-3′-methane sulfonyl-2-O-Me-thymidine.

In the final step, 5′-O-DMT-3′-hydroxy-2-O-Me-thymidine (450 mg, 0.81 mmol), CH₂Cl₂ (10 mL) and triethylamine (1.1 mL, 8 mmol) are reacted with methane sulfonyl chloride (308 uL, 4 mmol) to produce the precursor. The reaction was stirred for 3 hours at room temperature. The reaction was then concentrated onto Celite and purified by column chromatography using 5% MeOH:CH₂Cl₂ as the eluent to afford 450 mg (85%) of a clear, colorless foam.

¹H NMR (300 MHz, CD₃CN) δ: 1.76 (3H, s), 2.89 (3H, s), 3.34-3.37 (1H, m), 3.50-3.56 (1H, m), 3.79 (6H, s), 3.94 (3H, s), 4.35-4.38 (1H, m), 5.22-5.25 (1H, m), 6.11-6.15 (1H, dd, J=7.76, 2.5 Hz), 6.87-6.92 (4H, m), 7.27-7.39 (9H, m), 7.48-7.51 (2H, m).

¹³C NMR (75 MHz, CD₃CN) δ: 13.88, 38.73, 40.49, 55.91, 55.94, 62.67, 80.10, 82.93, 86.65, 87.54, 114.12, 117.24, 127.96, 128.89, 128.99, 131.01, 133.89, 136.59, 136.67, 145.79, 156.33, 159.79.

MS: Calc'd for C₃₃H₃₆N₂O₉S: 636.21; found: 637 (M+H).

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for preparing a compound having the following formula: 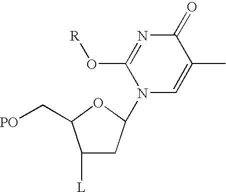
wherein R is an alkoxy blocking group; P is a hydroxyl protecting group; and L is a leaving group, the method comprising the steps of:
a. reacting a compound of the formula: 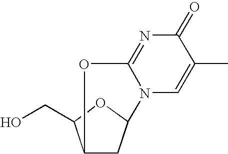
with a hydroxyl protecting group to produce a compound having the following formula: 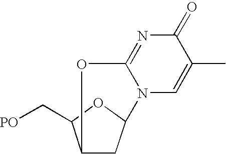
wherein P is the same as defined above;
b. enolating the reaction product of step (a) produce a compound having the following formula: 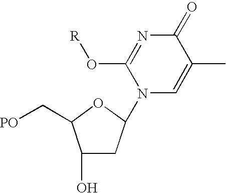
wherein P and R are the same as defined above; and
c. incorporating a leaving group to produce a compound having the following formula:

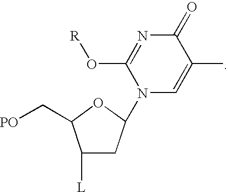

2. The method according to claim 1, wherein P is selected from the group consisting of methoxymethyl ether, methylthiomethyl ether, 2-methoxyethoxymethyl ether, 1-ethoxyethyl ether, 1-methyl-1-methoxyethyl ether, t-butyl ether, allyl ether, benzyl ether, 4-nitrobenzyl ether, o-nitrobenzyl ether, trityl ether, monomethoxytrityl ether, dimethoxytrityl ether, tritylone ether, tetrahydropyran ether, tetrahydrothiopyranyl ether, 4-methoxy tetrahydropyran ether, 4-methoxytetrahydrothiopyranyl ether, tetrahydrofuran ether, tetrahydrotriofuranyl ether, isobutyrate ester, pivaloate ester, adamantoate ester, benzoate ester, 2,4,6,-trimethylbenzoate ester, methyl carbonate, allyl carbonate, benzyl carbonate, p-nitrobenzyl carbonate, t-Bu carbonate, S-benzylthio carbonate, N-phenyl carbamate, and nitrate ester.

3. The method according to claim 1, wherein P is selected from the group consisting of dimethoxytrityl, monomethoxytrityl, trityl, t-butyloxycarbonyl, t-butyldimethylsilyl, t-butyldiphenylsilyl, tetrahydropyranyl ether, tetrahydrofuranyl ether, ethoxyethyl ether, and 1-methyl-1-methoxyethyl ether.

4. The method according to claim 1, wherein R is alkyl C₁-C₄, i-propyl, benzyl, cycloalkane C₃-C₆, phenyl, tosyl, acetate, or benzoate.

5. The method according to claim 1, wherein R is methyl, ethyl, i-propyl, benzyl, or cycloalkane C₃-C₆.

6. The method according to claim 1, wherein step (b) includes treating the reaction product of step (a) with an alkoxide having 1 to 4 carbons, cycloalkoxide C₃-C₆, phenoxide, tosyate, acetate, or benzoate.

7. The method according to claim 6, wherein the alkoxide is sodium methoxide.

8. The method according to claim 1, wherein L is a sulfonate ester.

9. The method according to claim 1, wherein L is selected from the group consisting of mesylate, nosylate, tosylate, and triflate.

10. A method for preparing a precursor for the preparation of a radiolabeled nucleoside comprising:
a. converting a 2-deoxy nucleoside into a 2,3′-anhydronucleoside;
b. reacting the 2,3′-anhydronucleoside with a hydroxyl protecting group to produce a 2,3′-anhydronucleoside derivative wherein the 5′-O group is protected;
c. reacting the protected 2,3′-anhydronucleoside derivative with a reagent that opens the 2,3′-anhydro-ring and enolates the 2-position on the pyrimidine ring; and
d. incorporating a leaving group to produce the radiolabeled nucleoside precursor.

11. The method according to claim 10, wherein the nucleoside is thymidine, cytidine, or uridine.

12. A method for preparing a precursor for the preparation of ¹⁸F-FLT comprising:
a. converting thymidine into 2,3′-anhydrothymidine;
b. reacting the 2,3′-anhydro thymidine with a hydroxyl protecting group to produce a 2,3′-anhydrothymidine derivative wherein the 5′-0 group is protected;
c. reacting the protected 2,3′-anhydrothymidine derivative with a reagent that opens the 2,3′-anhydro-ring and enolates the 2-position on the pyrimidine ring; and
d. incorporating a leaving group to produce the ¹⁸F-FLT precursor.

13. The method according to claim 12, wherein step (c) produces an enol having an —O—R group attached to the 2-carbon.

14. A method according to claim 13, wherein R is alkyl C₁-C₄, i-propyl, benzyl, cycloalkane C₃-C₆, phenyl, tosyl, acetate, or benzoate.

15. A method according to claim 12, wherein step (c) includes treating the reaction product of step (b) with an alkoxide.

16. A method according to claim 16, wherein the alkoxide is sodium methoxide, sodium ethoxide,

17. A method according to claim 12, wherein the hydroxyl protecting group is dimethoxytrityl, monomethoxytrityl, trityl, t-butyloxycarbonyl, t-butyldimethylsilyl, t-butyldiphenylsilyl, tetrahydropyranyl ether, tetrahydrofuranyl ether, ethoxyethyl ether, or 1-methyl-1-methoxyethyl ether.

18. A method according to claim 12, wherein the hydroxyl protecting group is dimethoxytrityl, monomethoxytrityl, or trityl.

19. A method according to claim 12 wherein the leaving group is a sulfonate ester.

20. A method according to claim 19, wherein the leaving group is mesylate, tosylate, nosylate, or triflate.

21. A compound having the following formula: 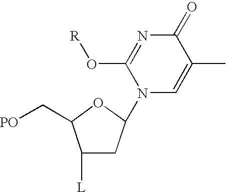
wherein R is alkyl C₁-C₄, i-propyl, benzyl, cycloalkane C₃-C₆, phenyl, tosyl, acetate, or benzoate; P is a hydroxyl protecting group; and L is a leaving group.

22. A compound according to claim 21, wherein R is methyl or ethyl.

23. A compound according to claim 21, wherein P is methoxymethyl ether, methylthiomethyl ether, 2-methoxyethoxymethyl ether, 1-ethoxyethyl ether, 1-methyl-1-methoxyethyl ether, t-butyl ether, allyl ether, benzyl ether, 4-nitrobenzyl ether, o-nitrobenzyl ether, trityl ether, monomethoxytrityl ether, dimethoxytrityl ether, tritylone ether; tetrahydropyran ether, tetrahydrothiopyranyl ether, 4-methoxy tetrahydropyran ether, 4-methoxytetrahydrothiopyranyl ether, tetrahydrofuran ether, tetrahydrotriofuranyl ether, isobutyrate ester, pivaloate ester, adamantoate ester, benzoate ester, 2,4,6,-trimethylbenzoate ester; methyl carbonate, allyl carbonate, benzyl carbonate, p-nitrobenzyl carbonate, t-Bu carbonate, S-benzylthio carbonate, N-phenyl carbamate, or nitrate ester.

24. A compound according to claim 21, wherein P is dimethoxytrityl, monomethoxytrityl, trityl, t-butyloxycarbonyl, t-butyldimethylsilyl, t-butyldiphenylsilyl, tetrahydropyranyl ether, tetrahydrofuranyl ether, ethoxyethyl ether, or 1-methyl-1-methoxyethyl ether.

25. A compound according to claim 21, wherein P is dimethoxytrityl.

26. A compound according to claim 21, wherein L is a sulfonate ester.

27. A compound according to claim 21, wherein L is selected from the group consisting of p-(2,4-dinitroanilino)benzenesulfonyl, benzenesulfonyl, methylsulfonyl (mesylate), p-methylbenzenesulfonyl (tosylate), 4-nitrobenzene sulfonyl (nosylate), p-bromobenzenesulfonyl, trifluoromethylsulfonyl (triflate), trichloroacetimidate, 2,2,2-trifluoroethanesulfonyl, imidazolesulfonyl.

28. A compound according to claim 21, wherein R is methyl, P is dimethoxy trityl, and L is mesylate, tosylate, or nosylate.

29. A compound having the following formula: 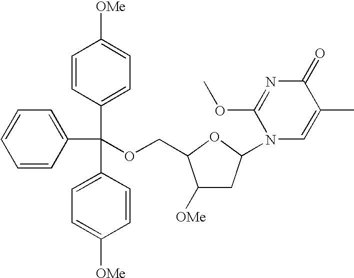
wherein Ms is methylsulfonyl.

30. A compound having the following formula: 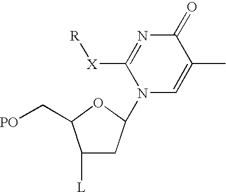
wherein R is alkyl C₁-C₄, i-propyl, benzyl, cycloalkane C₃-C₆, phenyl, tosyl, acetate, or benzoate; P is a hydroxyl protecting group; X is oxygen, sulfur, or nitrogen, and L is a leaving group.

31. A compound according to claim 30, wherein L is halogen, p-(2,4-dinitroanilino)benzenesulfonyl, benzenesulfonyl, methylsulfonyl (mesylate), p-methylbenzenesulfonyl (tosylate), 4-nitrobenzene sulfonyl (nosylate), p-bromobenzenesulfonyl, trifluoromethylsulfonyl (triflate), trichloroacetimidate, acyloxy, 2,2,2-trifluoroethanesulfonyl, imidazolesulfonyl, or 2,4,6-trichlorophenyl.

32. A compound according to claim 30, wherein P is dimethoxytrityl, monomethoxytrityl, trityl, t-butyloxycarbonyl, t-butyldimethylsilyl, t-butyldiphenylsilyl, tetrahydropyranyl ether, tetrahydrofuranyl ether, ethoxyethyl ether, or 1-methyl-1-methoxyethyl ether.

33. A method for preparing ¹⁸F-FLT comprising the steps of:
a. reacting a compound having the following formula: 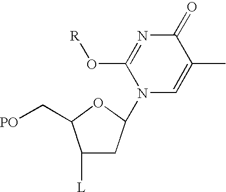
wherein R is alkyl having 1 to 4 carbons; P is a hydroxyl protecting group; and L is a leaving group, with [¹⁸F] to produce a compound having the following formula: 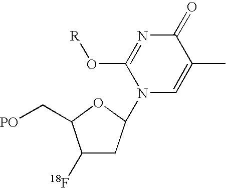
wherein R and P are the same as defined above; and
c. removing the alkyl group and protecting group to produce a compound having the following formula:

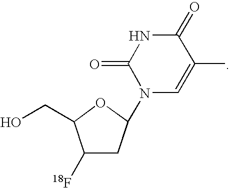

34. A method according to claim 33, wherein the step of removing the protecting group includes reacting the reaction product of step (b) with HCl, HBr, HOAc, H₂SO₄, HI, trimethylsilyliodide, or H₃PO₄.
